(19) United States
(12) Patent Application Publication
(10) Pub. No.: US 2004/0072744 A1
(43) Pub. Date: Apr. 15, 2004

(54) Synthetic peptide as treatment for down's syndrome and schizophrenia

(76) Inventors: Binie V. Lipps, Bellaire, TX (US); Frederick W. Lipps, Bellaire, TX (US)

Correspondence Address: John R. Casperson, PO Box 2174, Friendswood, TX, 77549, US

(21) Appl. No.: 10/242,175

(22) Filed: Sep. 12, 2002

(51) Int. Cl. A61K 38/08

(52) U.S. Cl. 514/12; 514/14; 514/15

(57) ABSTRACT

Adesh is a synthetic peptide consisting of at least the first four amino acids from the N-terminal of the sequence N L G E H P V C D S T D T W V (SEQ. ID. NO.: 1) and no more than 25 amino acids total. The synthetic peptide mimics the biological properties of nerve growth factor (NGF) consisting of 116 amino acids and is advocated to treat Down Syndrome (DS) and schizophrenia. It is believed that these neuro-degenerative diseases are linked with inadequate neurotrophic factors.

### Field of the invention

The invention relates to the use of a synthetic peptide to treat neurological disorders, especially Down's Syndrome and Schizophrenia.

### BACKGROUND OF THE INVENTION

Down's syndrome (DS) is a genetic condition of trisomy of chromosome 21, which is in triplicate. DS is named after Dr. John Langdon Down, an English physician who first described the characteristics of DS in 1866. It was not until 1959 that Jerome Leieune and Patricia Jacobs independently first determined the cause to be trisomy of the 21^{st} chromosome and 96% of DS individuals have chromosome 21 in triplicate. Among people with Down's syndrome, 3 to 4% of have Robertsonian Translocation, where the extra chromosome is attached with chromosome 14.

The production of excessive beta amyloid plaques and amyloid angeopathy in DS and Alzheimer's Disease (AD) is linked to a gene on human chromosome 21 that codes for beta-amyloid (Delabar et al. (1987) “Beta amyloid gene triplication in Alzheimer's disease and karyotypically normal Down syndrome. Science 235: 1390-1392). Because of this relationship between DS and AD, it has been assumed that knowledge about almost any aspect of one of these conditions will illuminate the other (Mann et al. (1984) “Alzheimer's presenile dementia, senile dementia of Alzheimer type and Down syndrome in middle age from an age related continuity of pathological changes” Neuropathol. Appl Neurobiol. 10: 185-207). However, DS has been largely untreatable. DS children are born with physical abnormality in development of eustachian tubes causing frequent ear infections. Structural abnormalities connected with the inadequate development of the entire maxillary region are very characteristic features of DS and responsible for many facial features associated with trisomy 21. Persons with DS have increased susceptibility to infection due to an immunoglobin subclass deficiency. Overall, respiratory infections remain a major problem. In addition, people with DS are at particularly high risk for AD, a progressive dementing disorder with characteristic clinical signs and brain pathology. DS victims are irritable, non-cooperative and suffer permanent defects in physical and verbal performance. The IQ of DS victims remains below 60.

A muscular seizure is a loss of control of the body by the brain. From 5 to 10% of DS children have seizure problems. Some children with DS have a constipation condition referred as Hirscprung disease. Some DS babies are born with heart defects, some with the associated illnesses such as epilepsy, hypothyroidism or celiac disease. Most individuals with DS have developmental disabilities, such as developmental delay, speech abnormalities, and degrees of cognitive dysfunction. Post mortem examination of DS victims brains revealed that they are smaller than that of normal people. The Hippocampus appears to be reduced to 30 to 50% (Crome (1972) “Pathology of mental retardation” 2nd Edition, Churchill Livingstone.)

Dementia is a symtomatological diagnosis in which there is loss of intellectual functioning severe enough to interfere with occupational or social functioning (American Psychiatric Association (1987). Psychological testing for dementia of individuals with mental retardation generally begins with an IQ test. Depressed people tend to complain about their lives and exaggerate their failures, where as individuals with AD tend to externalize their complains and to cover up their symptoms. The clinical diagnosis of dementia is difficult to make in individuals with DS. According to Katzman (1981) there are about 50 different causes of dementia in normal adults. Once dementia has been documented in DS individuals over the age of 30 years, the type must be established,—i.e., whether the dementia is a reversible condition such as reactive depression, endocrine and vitamin deficiencies, and uncontrolled seizures, or irreversible condition such as AD, subacute sclerotic panencephalitis, Creutzfeld-Jacob disease, AIDS or Huntington chorea. AD occurs in more than 50% of all cases of dementia that are associated with old age.

Genetic factors appear to play a significant role in both AD and DS. In addition, there is evidence for a familial link between AD and DS. Families with members who have died with AD have shown higher rates of births with DS (3.5/1000) versus families with no history of AD (1/1000) (Heston et al. (1981) “Dementia of the Alzheimer type. Clinical genetics, natural history and associated conditions” Arch. Gen. Psychiatry 38: 1085-1091.) A total of 26 gene loci on chromosome 21 have been identified in DS (Epstein (1986) “Trisomy 21 and nervous system from cause to cure” In Epstein C. J. (ed.) The neurobiology of DS New York, Raven p. 1.) The gene that codes β-amyloid to human chromosome 21 seems to be a direct relevance to the production of excessive amyloid beta-plaque and amyloid angeopathy in DS and AD providing a genetic link.

The genes on chromosome 21 having specific functions are: gene AD1 for Alzheimer disease (by linkage); gene APP for amyloid beta (A4) precursor protein; gene ALS for Amyotrophic Lateral Sclerosis; gene EPM1 for Progressive Myoclonus Epilepsy and the gene S100B for S100 protein, beta polypeptide, and glial specific protein associated with neurite outgrowth. One of the genes specific protein associated with neurite outgrowth is also located on chromosome 21. Lipps observed that DS people also have low level of NGF; therefore this gene may be silent or not functioning in them as well.

Nerve growth factor (NGF) was discovered half a century ago (Levi-Montalcini et al. 1954) “In vitro experiments on the effects of mouse sarcomas on spinal and sympathetic ganglia of the chick embryo” Cancer Res. 14: 49-57). NGF is a progenitor of a family of growth factors (Hamburger (1993) “The history of the discovery of the nerve growth factor” J. Neurobiol. 24: 893-897). Since then, several neurotrophic factors have been discovered, for example: neurotrophin-3 (NT-3), Neurotrophin-4/5, ciliary neurotrophic factor, (CNTF) and brain derived neurotrophic factor (BDNF). These factors are large protein molecules produced by neural cells to regulate nerve cell growth and survival. NGF, BDNF, NT-3, and NT-4/5 share approximately 50% amino acid sequence identity (Hallbrook (1991) “Evolutionary studies of nerve growth factor family revealed a novel member abundantly expressed in Xenopus ovary” Neuron 6: 845-858).

NGF is the prototypic neurotrophin that defines the properties and functions of the class of growth factors. NGF regulates the functions of the differentiated neurons. NGF is synthesized at a considerable distance from the cell body by peripheral tissues or other neurons that are contacted by exons of the NGF-sensitive neurons. Once the retrograde flow of NGF is established, it must continue for the life time of the neuron to develop and maintain the functional differentiated state of the neuron (Barde (1989) “Trophic factors and neuronal survival” Neuron 2:1525-1534).

Neurotrophic factor hypothesis states that developing neurons compete with each other for a limited supply of neurotrophic factor(s) provided by the target tissue. Successful competitors survive; unsuccessful ones die. Loss or inadequacy of neurotrophic factor/s may cause manifestation of schizophrenia, often in middle age, or AD, often in old age.

According to the mal developmental hypothesis, neurotrophins might be genetically and neurochemically involved in the etiopathogenesis of schizophrenia psychoses. (Thome et al. (1998) “Neurotrophic factors and the maldevelopmental hypothesis of schizophrenic psychoses” Review article. J. Neur. Trasm. 105: 85-100). Persons suffering from schizophrenia psychoses and control persons were genotyped for a null mutation of the ciliary neurotrophic factor gene and found no different. (Thome et al. (1997) “Ciliary neurotrophic factor null mutation and schizophrenic in Swedish population” Psychiatr. Genet. 7: 79-82). Preliminary clinical studies indicate that schizophrenia psychoses may be associated with changes in the genetic code of certain neurotrophic factors (Janssom et al. (1997) “Schizophrenia and Neurotrophin-3 alleles” Acta Psychiatr. Scand 414-419). Studies of brain anatomy and premorbid functioning indicate that schizophrenia psychoses may be of neurodevelopmental origin. It further supports for an association between the NT-3 gene and certain forms of schizophrenia psychoses (Jansson et al., ibid).

NGF is an important endogenous protein and its concentration varies under stress, infection and intoxication. Lipps reported the decreased levels of NGF in organs of mice as a consequence of sub-lethal injection of cobra venom. (Lipps (2002) “Decreased levels of nerve growth factor in organs of mice as a consequence of sub-lethal injection of cobra venom” J. Nat. Toxins 10: 283-290.) Lipps also discovered that the concentration of NGF in various organs of mice is age dependant. Furthermore, the presence of NGF is not restricted to cells of nervous system cells (Lipps, ibid).

Unlike all other organs, the blood-brain barrier regulates substances entering the brain via the blood circulation system. This barrier is a unique defense system, which shuts out most toxins; bacteria and viruses, which may be circulating in the blood, but allows the entry of necessary blood born molecules such as oxygen and glucose. Natural neurotrophic factors cannot cross the blood-brain barrier due to their size and therefore, cannot reach the brain when administered either orally or through injection.

It is imperative to discover small molecules having neurotrophic activity to overcome the blood-brain barrier. AIT-082 is the first compound made by Neotherapeutics that entered human clinical trials for neuro-degenerative diseases by oral administration. However, its efficacy is found to be marginal, showing no difference between Alzheimer's patients treated with AIT-082 and placebo. Glasky studied the effect of AIT-082 for working memory deficits associated with aging (Glasky et al. (1994) “Effect of AIT-082, a purin analog on working memory in normal and aged mice” Pharmacol Biochem Behav 47: 325-329). There are several publications regarding the synthetic NGF peptide derivatives which prevent neuronal death and show neurite outgrowth on rat adrenal pheochromocytoma (PC 12) cells, the characteristic of a neurotrophic factor (LeSautur et al. (1995) “Small peptide mimetics of nerve growth factor bind TrkA receptors and effect biological responses” J. Biol. Chem. 270: 6564-6569). It was reported that the peptides corresponding to the beta loop region of NGF were found to have the highest activity corresponding a loop region 29-35 which is capable to interact with the p75 receptor (Longo et al. (1993) “Nerve growth factor actions in PNS and CNS” In Laughlin S E, Fallon J H (eds) “Neurotrophic Factors” San Diego, Calif. Academic Press pp. 209-256; Longo, et al., (1997) “Synthetic NGF peptide derivatives present neuronal death via a p75 receptor dependant mechanism” J. Neuroscience Res. 48: 1-17).

The Ts65Dn mouse is segmental trisomic for a part of mouse chromosome 16 and it is a genetic model for Down syndrome and Alzheimer's disease (Hyde et al. (2001) “Ts65Dn mice a model for Down syndrome have deficits in context of discrimination learning suggesting impaired hippocampal function” Behav, Brain Res 118: 53-60). This mouse model is used to study certain developmental and degenerative abnormalities in the DS brain and TrkA is a receptor for NGF and its expression marks NGF-responsive CNS neurons and suggests novel roles for NGF in the brain (Holzman et al. (1995) “TrkA expression in the CNS: evidence for the existence of several NGF-responsive CNS neurons” J. Neurosci 15: 1567-76).

In recent years it has become evident that there is relationship between Alzheimer's disease (AD) and Down syndrome (DS), in which chromosome 21 is implicated. Preliminary clinical studies indicate that certain forms of schizophrenia may also be associated with changes in genetic code of neurotrophic factors.

### SUMMARY OF THE INVENTION

Adesh is a synthetic peptide consisting of at least the first four amino acids from the N-terminal of the sequence N L G E H P V C D S T D T W V (SEQ. ID. NO.: 1) and no more than 25 amino acids total. The synthetic peptide mimics the biological properties of nerve growth factor (NGF) isolated from naja naja venom consisting of 116 amino acids. (Lipps, B. V. et al., (2002) “Synthetic peptide for neurological disorders” WO 02/03922).

Adesh is advocated in this invention to treat Down Syndrome (DS) and schizophrenia. It is believed that these neuro-degenerative diseases are linked with the inadequate neurotrophic factors. Synthetic Adesh (Adesh-15) has been constructed using 15 amino acids N L G E H P V C D S T D T W V (SEQ. ID. NO.: 1), ten amino acids (Adesh-10) N L G E H P V C D S (SEQ. ID. NO.: 2), and five amino acids (Adesh-5) N L G E H (SEQ. ID. NO.: 3). The peptide Adesh can be made in abundance to provide therapeutic for Down syndrome and schizophrenia. Adesh has a low molecular weight (1,230 Daltons for Adesh-10) which enables it to overcome the blood-brain barrier and reach the brain when administered by many routes.

We have experimentally proved that Adesh-10 overcomes the blood brain barrier which is appropriate for a small synthetic analog of NGF having mol. wt. 1230 Daltons.

We have proved that Adesh mimics the biological properties of the natural NGF in cell cultures in regard to the replication of RNA viruses.

We are advocating Adesh as a therapeutic for the mental disorders such as Down's syndrome and schizophrenia.

### DETAILED DESCRIPTION OF THE INVENTION

Adesh-10 (SEQ. ID. NO.: 2) is considered exemplary of the Adesh family of peptides and has been studied and tested to illustrate the invention. Venom derived NGF produced neurites on PC12 cells at 5 ng/ml while the concentration of Adesh-10 required was 1 μg/ml. NGF is toxic to PC 12 cells at the concentration of 5 μg/ml, while Adesh-10 is not toxic up to 100 μg/ml.

From preclinical and clinical studies, it has become evident that it may be possible to use neurotrophic factors to prevent, slow the progression of the effects of neurodegenerative diseases and other types of insults in both the central nervous system (CNS) and the peripheral nervous system. However, the use of natural neurotrophins is hindered due to their large size. The cells of both CNS and the peripheral nervous systems are involved in neurological disorders. The molecules of any neurotrophin cannot be transported to the CNS due to the blood-brain barrier (BBB). The problem of transport across the BBB be may overcome by developing small-molecule NGF mimetics that maintain the neurotrophic activity of the protein while improving pharmacokinetics and disposition characteristics.

Adesh-10 is a synthetic peptide constructed by using the active domain of NGF consisting of ten amino acids. It is a small peptide having mol. wt. 1,230 Daltons, which mimics the biological properties of NGF. Adesh being a small molecule overcomes blood-brain-barrier. Thus, Adesh is an ideal therapeutic for the treatment for neurodegenerative disorders.

In the treatment method of the invention, a patient is identified as suffering from one of Down's Syndrome or Schizophrenia and Adesh peptide is administered to the patient. The peptide comprises at least the first four amino acids from the N-terminal of SEQ. ID. NO.: 1 and no more than 25 amino acids total. Preferably, the peptide contains in the range of 5 to 20 amino acids and is capable of crossing the blood-brain barrier and is administered to said patient in a manner to reach the bloodstream of the patient. Suitable administration techniques can be selected from the group consisting of nasal insufflation, buccal administration, oral ingestion, intramuscular injection and subcutaneous injection.

More preferably, the peptide contains in the range of 5 to 15 amino acids, and in the range of 0.01 to 10 milligrams of the peptide are administered on a daily basis. Even more preferably, in the range of 0.02 to 2 milligrams of the peptide are administered on a daily basis. In a most preferred embodiment, in the range of 0.05 to 1 milligrams of peptide is administered on a daily basis.

Where the condition to be treated is Down's Syndrome, the peptide preferably consists of SEQ. ID. NO.: 2 and in the range of 0.01 to 1.0 milligrams of the peptide are administered daily. Oral administration and subcutaneous injection of Adesh-10 at a dosage of about 0.1 milligrams/day has been tested with good results.

### EXPERIMENTAL AND RESULTS

In Vitro Cell Culture Experiments

Rotavirus and influenza virus require trypsin for their replication in cell cultures. It is reported here that trypsin can be replaced by NGF to grow these viruses. Therefore, the experiments were performed to compare the replication of Rotavirus and Influenza Virus in Medium supplemented with Trypsin, Nerve Growth Factor and Its synthetic Analog Adesh.

1. Comparison of Kinetics of Replication of SA11 Virus in MA104 Cells in Serum Free Medium Containing Trypsin, NGF or Adesh-10

Monolayers of African green monkey kidney cells (MA104) cells in 25 cm flasks were washed with serum free medium before infecting with simian rotavirus (SA11) at 10-2 dilution, having virus titer 10^{7.8}TCID₅₀/ml. After adsorption of the virus for 1.0 hour at 37° C. in humid CO₂ incubator, the inoculum was removed and the cultures were washed once with 2 ml of serum free medium. The infected cultures were divided into four groups. The flasks in group I received 4 ml of medium containing 1 μg/ml of trypsin, the flasks in group II received the medium supplemented with 1 μg/ml of NGF, the flasks in group III received 5 μg/ml of Adesh-10 and the flasks in group IV received medium containing equivalent amount of PBS. Two flasks from each group were harvested by freezing and thawing once, after 2, 4, 6 and 8 days of incubation. The pilot experiments were done several times. In the final experiment, harvests were assayed for infectivity in MA104 cells in triplicate and the titers were expressed as Log TCID₅₀/ml. Results are presented in Table I.

TABLE I
Kinetics of Replication of SA11 in MA104 cells
in serum free medium

| Medium | Day 2 | Day 4 | Day 6 | Day 8 |
|---|---|---|---|---|
| PBS | 2.1 | 3 | 3.5 | 4 |
| NGF | 4 | 7 | 7.5 | 8.2 |
| Adesh-10 | 3 | 6 | 6.5 | 8 |
| Trypsin | 6.5 | 7.2 | 8 | 8.5 |

Table 1 shows the replication of rotavirus SA11 strain in MA104 cells in the medium supplemented with 1 μg/ml trypsin, 1μ/ml of NGF and 5 μg/ml of Adesh-10. The virus yields at 2, 4, 6 and 8 days were higher in culture medium supplemented with trypsin, followed by NGF supplemented cultures. The cultures supplemented with Adesh-10 yielded lower virus yield at 2, 4 and 6 days. However, after 8 days incubation the virus yield was almost similar to that of NGF. The results show that the trypsin supplement used to grow rotavirus in the serum free medium can be replaced by similar concentration of NGF. Adesh-10 was supplemented in the serum free medium to compare the replication of rotavirus with NGF, as its additional biological property. In conclusion, the replication of rotavirus can be accomplished by supplementing the growth medium with NGF or Adesh-10.

2. Comparison of Kinetics of Replication of influenza A/TAI Virus in MDCK Cells in the Serum Free Medium Containing Trypsin, NGF or Adesh-10

The monolayers of Mandi-Darby canine kidney cells (MDCK) cells in 25 cm flasks were infected at 10⁻² dilution with influenza A virus isolated in Thailand in 1986 (A/ATI) influenza virus having 10^{6 5}TCID₅₀/ml. After adsorption of the virus for 1.0 hours at 37° C. in humid CO₂ incubator, the inoculum was removed and the cultures were washed once with 2 ml serum free medium. The infected cultures were divided into four groups. Four groups of infected flasks received medium and the supplements as described for rotavirus. Two flasks from each group were harvested by freezing and thawing once, after 2, 4 and 6 days of incubation. The pilot experiments were done several times. In the final experiment, harvests were assayed for infectivity in MDCK cells in triplicate and the titers were expressed as Log TCID₅/ml. Results are presented in Table II.

TABLE II
Kinetics of Replication of A/TA1 in Dog kidney cell
in serum free medium

| Medium | Day 2 | Day 4 | Day 6 |
|---|---|---|---|
| PBS | 3 | 4.1 | 4.3 |
| NGF | 5 | 5.5 | 6.1 |
| Adesh-10 | 4 | 5.1 | 6.2 |
| Trypsin | 5.6 | 6 | 6.5 |

Table II shows the replication of influenza virus A/TAI strain in MDCK cells in the medium supplemented with 1 μg/ml trypsin, 1 μg/ml of NGF and 5 μg/ml of Adesh-10. The virus yields at 2 and 4 days were higher in culture medium supplemented with trypsin, followed by lesser yields for NGF supplemented cultures. The cultures supplemented with Adesh-10 yielded lower virus at 2 and 4 days. However, after 6 days incubation, the virus yield was a little higher than that of NGF. The results show that the trypsin supplement used to grow influenza virus in the serum free medium can be replaced by similar concentration of NGF. Adesh-10 was supplemented in the serum free medium to compare the replication of influenza with NGF, as its additional biological property. In conclusion, the replication of influenza virus can be accomplished by supplementing the growth medium with NGF or Adesh-10.

In Vivo Experiments

One year old Balb/c male mice (considered to be retired and non-productive) were given Adesh-10 orally for seven consecutive days, and the control set of mice received PBS. Two days after the completion of the treatment, the mice were sacrificed for organs. Organ homogenates were assayed for NGF content by simple enzyme-linked immunosorbent assay (ELISA), using anti-NGF.

Preparation of organ homogenates: Mice were used in compliance with U.S. Public Health Service policy on humane care and use of animals. One-year-old Balb/c male mice were divided into two groups each consisting of five. Mice in group I were given orally 100 μg/mouse in 50 μl volume of Adesh-10, the synthetic analog of NGF for seven consecutive days. The group II mice were given similar volume of PBS. Two days after the completion of the treatment with Adesh-10 or PBS the mice were sacrificed for organs. Organs from five of each group of mice were collected and one type of organ from five were pooled. The pool of the organs was homogenized in PBS by manual homogenizer. The homogenates were centrifuged and the supernatants for each pool organ were separated. Protein concentration for each supernatant was measured on spectrophotometer using a protein kit from Bio-Rad (USA catalogue 500-0006). The protein concentrations of the supernatants were adjusted to 100 μg/ml with PBS, as stocks for further testing.

Enzyme-linked immunosorbent assay (ELISA): ELISA tests were carried out in 96 well microtiter plates. The reagents for ELISA were purchased from Sigma-Aldrich Co. The stocks from mouse organs were diluted with carbonate-bicarbonate buffer of pH 9.4 to 10 μg/ml as coating antigen for ELISA. The wells of the plate were coated with antigen, 100 μl/well. The plate was left at room temperature (RT) overnight, after which it was emptied and washed three times (3×) with PBS. The wells were blocked with 250 μl/well of 3% fish skin gelatin. After 30 min. the plate was emptied and washed 3× with PBS. Anti-NGF made in rabbit was diluted from 1:300 in gelatin. Three wells received 100 μl of each dilution of anti-serum. Two-fold diluted anti-sera were used from 1:300 in triplicate. The antigen controls without antibody were incorporated. The plate was incubated at 37° C. in a humid incubator for 1 hr after which the plate was washed 3× with PBS. The horseradish peroxidase conjugated with rabbit IgG 100 μl/well were reacted, for 30 min. Finally, after washing, the wells of the plate were reacted with O-phenylenediamine-HCl (OPD) for color development. The plate was read after 30 min. and the OD was recorded at 405 mm. The average of triplicate readings was expressed as ELISA titer/100 μl.

One year old Balb/c male mice were used for this research. Ten mice were used consisting of two groups five of each. Mice in group I were given orally 100 μg/mouse in 50 μl volume of Adesh-10, synthetic analog of NGF for seven consecutive days. The group II mice were given similar volume of PBS. Two days after the completion of the treatment the mice were sacrificed for organs. Organ homogenates were assayed for NGF by ELISA. The results are presented in Table III.

TABLE III
NGF concentration in organs of male mice expressed as
ELISA titer/100 μl.

| Organ | Control | Treated |
|---|---|---|
| Bone | 600 | 900 |
| Brain | 1200 | 2400 |
| Heart | 1800 | 2400 |
| Kidney | 1200 | 1800 |
| Liver | 1800 | 2400 |
| Lung | 1200 | 1800 |
| Muscle | 1200 | 2400 |
| Pancreas | 900 | 1200 |
| Salivary | 900 | 1200 |
| Skin | 1200 | 1200 |
| Spleen | 1200 | 1200 |
| Testis | 900 | 1800 |

Results in Table III show that the NGF content in the organs of control mice is much lower than the corresponding organs of mice treated with Adesh-10. There was twofold increase in NGF level in brain, muscle and testis. The ELISA titer/100 μl increased from 1200 to 2400 for brain and muscle and increased from 900 to 1800 in case of testis. The ELISA titer/100 μl for NGF was 24300.

The data show an increase in NGF level in organs of mice treated with Adesh-10, except for skin and spleen, in comparison to the control mice. The most increase in NGF levels was observed in brain, muscle and testis of mice treated with Adesh-10 in comparison to the controls. This clearly confirms that a small molecule like Adesh-10 having a molecular weight 1,230 Daltons was able to pass through the blood-brain barrier, to reach the brain, excite the neurons to produce more NGF.

Thus, it was revealed that there was increased level of NGF in all organs of mice treated with Adesh-10, in comparison to the controls. The most increased in NGF level was observed in brain, muscle and testis. Such experiments with oral administration of natural NGF cannot be performed due to blood-brain barrier. Therefore, Adesh is advocated for the treatment of various neurological disorders by oral route to increase the NGF level to normal homeostasis. Lipps reported that in mice the level of NGF goes down by age. By providing a treatment with Adesh may be causing reversal in aging or controlling of aging, at least in the nervous system.

In Humans: Several Down syndrome children were treated with Adesh-10. Adesh-10 was administered in the amount of 100 μg/day, either orally under the tongue, or by sub cutaneous injection. Down's syndrome children treated with Adesh-10 showed improved physical, vocal, learning and memory abilities.

The nervous system is the most complex system in human body. It consists of the central nervous system including the brain and the network of the peripheral nervous system. The neuron cells of both systems are implicated in the production of neurotrophins, which are anticipated in many processes such as proliferation, differentiation, migration, survival and synapse formation. The ability of neurotrophic factors to promote the survival of peripheral and central neurons during development and after neuronal damage, these molecules can be as potential therapeutics for the treatment of nerve injuries, neuro-degenerative diseases and also remodeling in psychiatric illness. It has shown that neurotrophins protect against neuronal dysfunction and death in animal models of injury and neurological diseases (Yuen et al.(1996) “Therapeutic potential of neurotrophic factors for neurological disorders” Ann. Neurol. 40: 346-354).

There is no mention of psychology in the Bible. However, it has been known for centuries that some individuals are born with a good mind and others with a bad mind, and there are also environmental factors. In my opinion the manifestation of adverse mind is considered to be psychological by people of advanced society. Schizophrenic psychosis is a change in the mind due to a decreased concentration of neurotrophins, leading to an imbalanced homeostasis in the nervous system (Thome et al. (1998) supra). Different types of insults can cause a change in the concentration of neurotrophin and imbalanced in homeostasis. The insults can be as follows:

1. Physical trauma; accident, fall etc.

2. Chemical insult from environment, drugs, medications etc.

3. Emotional insults are most complicated not well understood. Emotional insults can be due to disappointment, failure, a tragic event, fear, or even bad dreams.

These insults do not concern the people who commit crimes or wrong actions by pre-planning. Such pre-planned crimes or wrong actions aught to be due to a bad mind. Due to any of these types of insults some neurons get affected and cease to function normally, leading to a shadow or a clouded mind. The clouded mind or the shadow is termed as a psychological condition or schizophrenia by the modern world. During such psychological impact, some of the neurons are affected ceasing the functional neurotrophin production. The neurological insults may be manifested into jealousy, anger, grief, in some cases violent crime. Some of these manifestations, if temporary, may be reverted by taking a hot water bath, exercise, alcoholic drink or some kind of entertainment for diversion.

However, in some cases it can become worse because the affected neurons become non functional and die off. Currently, there is no specific treatment for mental illness including schizophrenia psychoses. Therefore, if such condition is treated with Adesh, the affected neurons can be activated and brought to normal state.

Normal people loose 0.2% brain volume per year whereas in AD the decrease is greater. This is a clear indication that the death of neurons causing decrease level of Neurotrophin is manifesting to AD. As we age, the neuron population goes down decreasing the Neurotrophin concentration. Theoretically, aging can be controlled by Neurotrophins treatment. However, since these are typically large in size, overcoming the blood-brain barrier will be a problem. On the other hand, Adesh, being a small molecule, will overcome the BBB. Normal people treated with Adesh, beside controlling aging, will not contract AD and other neurological diseases, including psychological disorders.

Thus Adesh treatment is advocated for the following:

1. Treatment for Alzheimer's Disease and the prevention and/or treatment of neurological disorders in general.

2. Treatment of Down's Syndrome

3. Treatment of Schizophrenia Psychoses

4. Control of Aging

While certain preferred embodiments of the invention have been described herein, the invention is not to be construed as being so limited, except to the extent that such limitations are found in the claims.

1. The invention discovered that individuals having Down syndrome show low level of nerve growth factor (NGF).

2. Synthetic Adesh consisting of SEQ. ID. NO.: 2 is advocated as a treatment for neurological disorders; Down syndrome and schizophrenia.

3. Composition of synthetic Adesh as a treatment for people who are diagnosed for Down syndrome and schizophrenia. Adesh treatment increases the NGF level which improves the physical and cognitive symptoms in DS victims.

4. The composition of synthetic Adesh as set forth in SEQ. I.D. NO.: 2.

5. The composition of claim 4 wherein the peptide contains the sequence of at least first four amino acids beginning at its N-terminal of SEQ. I.D. NO.: 1 and has no more than 20 amino acids total.

6. A method of using a composition as in claim 5 wherein the peptide is orally or subcutaneously (SC) administered to elevate NGF level and to improve symptoms.

7. The method in claim 6 wherein the in the range of from about 0.01 to about 10 milligrams of the peptide are orally or SC administered on a daily basis.

8. A method in claim 6 wherein in the range of from 0.02 to 2 milligrams of the peptide are orally or SC administered on a daily basis.

9. A method as in claim 8 wherein the peptide is selected from the group of consisting of minimum four amino acids and no more than twenty amino acids from N-terminal.

10. A method for treating a condition selected from the group consisting of Down's Syndrome and Schizophrenia in humans, comprising
identifying a patient suffering from one of said conditions, and
administering a peptide to said patient;
wherein the peptide comprises at least the first four amino acids from the N-terminal of SEQ. ID. NO.: 1 and no more than 25 amino acids total.

11. A method as in claim 10 wherein
the peptide contains in the range of 5 to 20 amino acids and is capable of crossing the blood-brain barrier, and
the peptide is administered to said patient in a manner to reach the bloodstream of the patient.

12. A method as in claim 11 wherein the peptide is administered by an administration technique selected from the group consisting of nasal insufflation, buccal administration, oral ingestion, intramuscular injection and subcutaneous injection.

13. A method as in claim 12 wherein
the peptide contains in the range of 5 to 15 amino acids.

14. A method as in claim 13 wherein in the range of 0.01 to 10 milligrams of the peptide are administered on a daily basis.

15. A method as in claim 14 wherein the condition is Down's Syndrome.

16. A method as in claim 15 wherein the peptide consists of SEQ. ID. NO.: 2 and in the range of 0.02 to 2 milligrams of the peptide are administered daily.

17. A method as in claim 16 wherein the peptide is administered orally or by subcutaneous injection.


SEQUENCE LISTING (3 sequences)

SEQ ID NO: 1 - protein, 15 aa, ARTIFICIAL
  CORRESPONDS TO FRAGMENT OF ISOLATE FROM NAJA NAJA VENOM.  SEE WO 02/03922.
NLGEHPVCDSTDTWV

SEQ ID NO: 2 - protein, 10 aa, ARTIFICIAL
  CORRESPONDS TO POSITIONS 1-10 OF SEQ. ID. NO. 1
NLGEHPVCDS

SEQ ID NO: 3 - protein, 5 aa, ARTIFICIAL
  CORRESPONDS TO POSITIONS 1-5 OF SEQ. ID. NO. 1
NLGEH